(19) United States
(12) Patent Application Publication
(10) Pub. No.: US 2023/0125827 A1
(43) Pub. Date: Apr. 27, 2023

(54) SYSTEMS AND METHODS FOR PRODUCING MIXTURES

(71) Applicants: Boston Scientific Medical Device Limited, Galway (IE); Boston Scientific Scimed, Inc., Maple Grove, MN (US)

(72) Inventors: Benjamin CLEVELAND, Bellingham, MA (US); Nitesh Ghananil BAVISKAR, Kalyan West (IN); Junaid Mohammed SHAIKH, Surat (IN); Richard Earl GRAFFAM, Pelham, NH (US); Joseph HERNANDEZ, Rutland, MA (US); Christopher WATSON, Lincoln, MA (US); Jennie CREEGAN, Minneapolis, MN (US); Kolbein KOLSTE, Acton, MA (US); Nicholas DeSANTIS, Cambridge, MA (US); Eric KIM, Brighton, MA (US); Mei Lee AMEND, Lakeville, MA (US); Subodh MOREY, Ponda (IN)

(73) Assignees: Boston Scientific Medical Device Limited, Galway (IE); Boston Scientific Scimed, Inc., Maple Grove, MN (US)

(21) Appl. No.: 18/048,494

(22) Filed: Oct. 21, 2022

Related U.S. Application Data
(60) Provisional application No. 63/262,956, filed on Oct. 22, 2021.

(51) Int. Cl. A61J 1/20

(52) U.S. Cl. CPC A61J 1/2096; A61J 1/2058

(57) ABSTRACT

A system for producing a mixture to deliver to a treatment site. A multi-lumen chamber can be connected to a proximal end of a mixing lumen and include a first lumen aligned and adjacent a second lumen. The first lumen can be configured to include a first constituent in a proximal portion of the first lumen and a second constituent in a distal portion of the first lumen. A first plunger can be internally positioned within the first lumen to distally move the first constituent into the distal portion to mix with the second constituent in a first state to form a first mixture. The first lumen can terminate in a first port. The second lumen is configured to include a third constituent. A second plunger is internally positioned within the second lumen to distally move the third constituent and the first mixture.

### CROSS-REFERENCE TO RELATED APPLICATIONS

This patent application claims the benefit of priority under 35 U.S.C. § 119 to U.S. Provisional Patent Application No. 63/262,956, filed Oct. 22, 2021, which is herein incorporated by reference in its entirety.

### FIELD

The present disclosure relates generally to compositions for injection to a patient, methods of preparation and use thereof, and devices comprising such compositions.

### BACKGROUND

Numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Since the conception of conformal radiotherapy, physicians have paid attention to the delivered dose to the target and surrounding tissues. Investigators have been able to correlate side effects to the amount of tissue receiving a certain radiation dose. And yet, time, distance, and shielding affect the dose that is delivered. The less time an area is exposed to radiation, the less dose delivered. The greater the distance from the radiation, the less dose delivered.

Current systems provide filler material to treatment sites to decrease the radiation dose to the rectum during radiotherapy for prostate cancer. However, the system that mixes the filler material in vitro includes numerous subcomponents, is complex to assemble, and rife with filler mixing errors prior to delivery within a patient at a treatment site. During the foregoing procedures, such errors and mishaps lead unnecessarily to patient risk, increased procedure time, and increased procedure costs. The solution of this disclosure resolves these and other issues of the art.

### SUMMARY

In accordance with certain aspects of the present disclosure, a system is disclosed for producing a mixture to deliver to a treatment site. The system can include a multi-lumen chamber connected to a proximal end of a mixing lumen that includes a first lumen aligned and adjacent a second lumen. The first lumen can be configured to include a first constituent in a proximal portion of the first lumen and a second constituent in a distal portion of the first lumen. A first plunger can be internally positioned within the first lumen to distally move the first constituent into the distal portion to mix with the second constituent in a first state to form a first mixture. The first lumen can terminate in a first port. The second lumen can be configured to include a third constituent. A second plunger can be internally positioned within the second lumen to distally move the third constituent and the first mixture. The second lumen can terminate in a second port. Distally moving the second plunger can cause the first mixture and the third constituent to be delivered through the first and second ports and mixed together within the mixing lumen to form the mixture.

In accordance with certain aspects of the present disclosure, a connector can be included that has the mixing lumen and is attachable to a distal end of the multi-lumen chamber and a proximal end of a needle. The system is configured to deliver the mixture through the mixing lumen and the needle.

In accordance with certain aspects of the present disclosure, the needle is connected to a distal end of the connector.

In accordance with certain aspects of the present disclosure, the mixing lumen of the connector includes a static mixer.

In accordance with certain aspects of the present disclosure, the connector includes a greatest width adjacent a distal end of the multi-lumen chamber, and wherein at least one of the first port and the second port includes an air-permeable fluid-impermeable membrane.

In accordance with certain aspects of the present disclosure, the connector further including a first tube configured to be in fluid communication with the first port and the mixing lumen of the connector. A second tube can be included and configured to be in fluid communication with the second port and the mixing lumen of the connector.

In accordance with certain aspects of the present disclosure, the first and second tubes together form a Y-shape.

In accordance with certain aspects of the present disclosure, proximal and distal ends of the first and second lumens are aligned with each other in a second state.

In accordance with certain aspects of the present disclosure, the first constituent is a diluent and the third constituent is an accelerator.

In accordance with certain aspects of the present disclosure, the first mixture is a precursor fluid solution.

In accordance with certain aspects of the present disclosure, the proximal and distal portions of the first lumen are separated by a barrier. Distally moving the first plunger causes the barrier to open so the first constituent mixes with the second constituent in the first state to form the first mixture.

In accordance with certain aspects of the present disclosure, the proximal portion of the first lumen. The first plunger and the second plunger are integrally formed in a plunger assembly.

In accordance with certain aspects of the present disclosure, the first plunger is configured to advance within the first lumen independent of the second plunger.

In accordance with certain aspects of the present disclosure, the second lumen and the distal portion of the first lumen are positioned within a lumen receiver. In a second state, the first and second plungers are at least partially positioned within the lumen receiver.

In accordance with certain aspects of the present disclosure, in a first state, the system further includes a retainer removably positioned between a flange of the second plunger and the multi-lumen chamber so as to prevent movement of the second plunger. In a second state, the retainer is removed so that the second plunger is capable of distally moving the second plunger to cause at least one of the first mixture and the third constituent to egress through a respective lumen and mix together in the mixing lumen to form the mixture.

In accordance with certain aspects of the present disclosure, a connector is disclosed for mixing and delivering a mixture to a treatment site. The distal end of the connector is configured to be attached to a needle. A proximal end and be wider than the distal end. A mixing lumen can be between the proximal and distal ends and extended proximally from the distal end and configured to be in fluid communication with a lumen of a needle. A first tube can be proximally extended from a proximal end of the mixing lumen to a first lumen receiver. A second tube can be proximally extended from the proximal end of the mixing lumen and opposite the first tube to a second lumen receiver.

In accordance with certain aspects of the present disclosure, the first and second tubes form a Y-shape.

In accordance with certain aspects of the present disclosure, the mixing lumen includes a static mixer.

In accordance with certain aspects of the present disclosure, an outer surface of the connector is tapered between proximal and distal ends.

In accordance with certain aspects of the present disclosure, a proximal end of the mixing lumen is positioned centrally within an inner diameter of the connector between the proximal end distal ends of the connector.

In accordance with certain aspects of the present disclosure, a method for producing a mixture with a mixing system to deliver to a treatment site. The mixing system can include a mixing lumen and a multi-lumen chamber connected to a proximal end of the mixing lumen including a first lumen aligned and adjacent a second lumen. The first lumen can be configured to include a first constituent in a proximal portion of the first lumen and a second constituent in a distal portion of the first lumen. A first plunger can be internally positioned within the first lumen and terminating in a first port. The second lumen can be configured to include a third constituent. The second plunger can be internally positioned within the second lumen and terminating in a second port. The method can include distally moving the first constituent, by the first plunger, to open a barrier within the first lumen thereby injecting the first constituent into the distal portion to mix with the second constituent in a first state to form a first mixture; and distally moving the second plunger causing the first mixture to expel from the first port and the third constituent to expel from the second port and mixed together within the mixing lumen to form the mixture.

In accordance with certain aspects of the present disclosure, the method can include connecting a primed connector and a needle to a distal end of the multi-lumen chamber, the primed connector including the mixing lumen and being a Y-shaped connector. The method can include positioning a first tube in fluid communication with the first port and the mixing lumen of the primed connector; and positioning a second tube in fluid communication with the second port and the mixing lumen of the primed connector.

In accordance with certain aspects of the present disclosure, the proximal portion of the first lumen, the first plunger and the second plunger are integrally formed in a plunger assembly. The method can include positioning the second lumen and the distal portion of the first lumen within a lumen receiver; and at least partially positioning the first and second plungers within the lumen receiver in a second state.

In accordance with certain aspects of the present disclosure, the first constituent is a diluent and the third constituent is an accelerator, and wherein the first mixture is a precursor fluid solution.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIGS. 1A-1B depict the prostate, rectum, and Denonvilliers' space between the prostate and rectum.

FIG. 2 shows an exploded view of an exemplary mixing system in accordance with certain aspects of the present disclosure.

FIG. 3 shows an exploded view of an exemplary mixing system in accordance with certain aspects of the present disclosure.

FIG. 4 depicts a plunger assembly in accordance with certain aspects of the present disclosure.

FIG. 5 depicts a partial cross-section view of an example connector and needle attached to an example mixing system, in accordance with certain aspects of the present disclosure.

FIGS. 6A-6B depict example steps in a method using an example mixing system, in accordance with certain aspects of the present disclosure.

FIGS. 7A-7B depict example steps in a method using an example mixing system, in accordance with certain aspects of the present disclosure.

FIGS. 8A-8C depict example steps in a method of priming an example connector, in accordance with certain aspects of the present disclosure.

FIGS. 9A-9B depict example steps in a method using an example mixing system and a primed connector, in accordance with certain aspects of the present disclosure.

FIG. 10 depicts a flow diagram of a method of using a mixing system according to certain aspects of this disclosure.

### DETAILED DESCRIPTION

Particular aspects of the present disclosure are described in greater detail below. The terms and definitions provided herein control, if in conflict with terms and/or definitions incorporated by reference.

Particular aspects of the present disclosure may repeat reference numerals and/or letters in the various examples. This repetition is for the purpose of simplicity and clarity and does not in itself dictate a relationship between the various embodiments and/or configurations discussed. Different embodiments may have different advantages, and no particular advantage is necessarily required of any embodiment.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise.

As used herein, “approximately” and “about” refer to being nearly the same as a referenced number or value. As used herein, the terms “approximately” and “about” should be understood to encompass ±10% of a specified amount or value (e.g., “about 90%” can refer to the range of values from 81% to 99%).

As used herein, “operator” can include a doctor, surgeon, or any other individual or delivery instrumentation associated with delivery or use of a mixing system as such systems are described throughout this disclosure.

The compositions herein may be used in various medical procedures, including but not limited to injected to create additional space between the rectum and prostate during treatment, for example in the Denonvilliers' space, thereby reducing rectal radiation dose and associated side effects. Certain embodiments of the disclosure include placing a filler between the radiation target tissue and other tissues. The filler can be a gel composition that increases the distance between the target tissue and other tissues so that the other tissues receive less radiation.

It is understood that “Denonvilliers' space” is a region located between the rectum and prostate. Certain embodiments provide a method of displacing a tissue to protect the tissue against the effects of a treatment involving radiation or cryotherapy. One embodiment involves using a filler mixed by a mixing system of this disclosure to displace the tissue relative to a tissue that is to receive the treatment. Another embodiment involves introducing a filler mixed by a mixing system of this disclosure to displace a first tissue and radiating a second tissue, particularly a second tissue that is close to the first tissue. In another embodiment, the method includes the steps of injecting a filler into a space between tissues; and may further include irradiating one of the tissues so that the other tissue receives less radiation than it would have in the absence of the filler.

Certain embodiments also provide methods for treating a tissue of a body by radiation. In one embodiment, the method includes the steps of injecting an effective amount of a filler into a space between a first tissue (e.g., prostate) of a body and a second tissue (e.g., rectum), which can be a critically sensitive organ; and treating the first tissue by radiation whereby the filler within the space reduces passage of radiation into the second tissue. Tissue is a broad term that encompasses a portion of a body: for example, a group of cells, a group of cells and interstitial matter, an organ, a portion of an organ, or an anatomical portion of a body, e.g., a rectum, ovary, prostate, nerve, cartilage, bone, brain, or portion thereof.

The gel of the filler can include polymeric materials which are capable of forming a hydrogel may be utilized. In one embodiment, the polymer forms a hydrogel within the body. A hydrogel is defined as a substance formed when an organic polymer (natural or synthetic) is crosslinked via covalent, ionic, or hydrogen bonds to create a three-dimensional open-lattice structure which entraps water molecules to a gel. Naturally occurring and synthetic hydrogel forming polymers, polymer mixtures, and copolymers may be utilized as hydrogel precursors.

In some aspects, the hydrogel can be formed by a composition formed by mixing constituents together (E.g., accelerant fluid, diluent, and PEG together) and may comprise one or more polysaccharide compounds or a salt thereof. For example, the composition may include a cellulose compound such as carboxymethyl cellulose (CMC) or salt thereof (e.g., CMC) sodium, xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be crosslinked with a suitable crosslinking compound, such as butanediol diglycidyl ether (BDDE). In some aspects, the polysaccharide may be a homopolysaccharide or a heteropolysaccharide

The present disclosure also provides mixing systems to form the gel composition and corresponding medical devices for use and/or delivery to a treatment site of a patient. According to some aspects of the present disclosure, the mixing system may include a plurality of reservoirs with respective lumens. Collectively, the lumens therein may serve as a container for constituents to mix the gel composition of this disclosure. Suitable reservoirs may include, for example, syringes (e.g., a syringe barrel compatible with a manual or automatic injection system) and other fluid containers configured for use with a suitable injection needle. Exemplary materials suitable for the reservoir include, but are not limited to, cyclic olefin polymer, polypropylene, polycarbonate, polyvinyl chloride, and glass. In some aspects, one of these materials (e.g., cyclic olefin copolymer specifically) can have a coating applied to it, such as SiO₂), which is advantageous so the coating can perform as a primary oxygen barrier, behave as a glass-like layer, and can be applied using a vapor deposition process.

According to some aspects of the present disclosure, the compositions may include at least one accelerant (e.g., an activating agent) combined with a precursor mixed from a diluent (e.g., mostly water) and polyethylene glycol (PEG). In some examples, the composition may be or include a gel with a desired gel strength and/or viscosity, such as a biocompatible gel suitable for injection (e.g., through a needle).

The hydrophilic polymer can be any gelling agent(s), including natural ones or synthetic in origin, and may be anionic, cationic, or neutral. Non-limiting examples of the gelling agents include polysaccharides such as gellan gum, xanthan gum, gum arabic, guar gum, locust bean gum, alginate, and carrageenans.

The concentrations of gelling agent(s) in the composition described in this disclosure may range from about 0.01% to about 2.0% by weight with respect to the total weight of the composition, such as from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, the composition may have a viscosity ranging from about 0.001 pascal-second (Pa·s) to about 0.100 Pa·s at a shear rate of 130 s⁻¹, such as, e.g., from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, the composition may have a viscosity ranging from about 0.001 Pa·s to about 0.050 Pa·s at a shear rate of 768 s⁻¹, such as, e.g., from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The mixing system herein may include or be removably connected to one or more needles. In some examples, the needle may be a hypodermic needle, and may range from a size of 7 gauge (4.57 mm outer diameter (OD), 3.81 mm inner diameter (ID)) to 33 gauge (0.18 mm OD, 0.08 mm ID), e.g., a size of 16 gauge (1.65 mm OD, 1.19 mm ID), 18 gauge, 21 gauge (0.82 mm OD, 0.51 mm ID), 22 gauge (0.72 mm OD, 0.41 mm ID), 23 gauge (0.64 mm OD, 0.33 ID), or 24 gauge (0.57 mm OD, 0.31 mm ID). Exemplary materials for the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

According to some aspects of the present disclosure, the filler compositions herein, e.g., the compositions prepared by the methods herein may have sufficient strength, e.g., gel strength, to withstand the forces and thus minimizing the effects of the forces on the continuity of the three-dimensional gel network. In the meantime, the composition with sufficient strength may have a viscosity suitable for injection, e.g., a viscosity that does not render the composition stuck in the reservoir(s), delivery lumen, or a needle connected therewith.

According to some aspects of the present disclosure, the composition may maintain its three-dimensional structure until the gel is injected through a needle, whereupon the structure may form fragments of the original continuous, three-dimensional network. Those gel fragments may have a diameter corresponding to the diameter of the injection needle, such that the fragments are as large as possible in-vivo to retain as much of the three-dimensional structure of the gel as possible. Injection of these larger-sized particles or fragments is believed to increase the amount of time the gel remains within the tissue.

The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lbf to about 25 lbf, such as from about 10 lbf to about 20 lbf, e.g., about 15 lbf. The loads measured for a given gel concentration may vary for different needles and flow rates.

According to some aspects of the present disclosure, the size of the needle may be chosen based on the viscosity and/or components of the composition, or vice versa. According to some aspects of the present disclosure, the size of the needle may be 23 gauge or 25 gauge. In some cases, a larger size of 18-gauge, 20 gauge, 21 gauge, or 22 gauge may be used to inject the compositions herein.

According to some aspects of the present disclosure, the mixing system of this disclosure can be included in a kit for introducing a filler into a patient, whereby the filler can include any of the gel compositions of this disclosure. Kits or systems for mixing a gel composition of this disclosure, such as hydrogels, may be prepared so that the precursor(s) and any related activating agent(s) are stored in the kit with diluents as may be needed. Applicators may be used in combination with the same. The kits can be manufactured using medically acceptable conditions and contain components that have sterility, purity and preparation that is pharmaceutically acceptable. Solvents/solutions may be provided in the kit or separately. The kit may include syringes and/or needles for mixing and/or delivery. The kit or system may comprise components set forth herein.

During some examples of use, once saline has been injected to the treatment site, a mixing system can be connected to a needle (e.g., an 18-gauge spinal needle) to then inject a 5-10 mm layer of filler (e.g., gel composition) along the posterior wall of the prostate between the prostate and rectum. Once the filler has been injected into the space between the rectum and prostate, ultrasound images can be obtained.

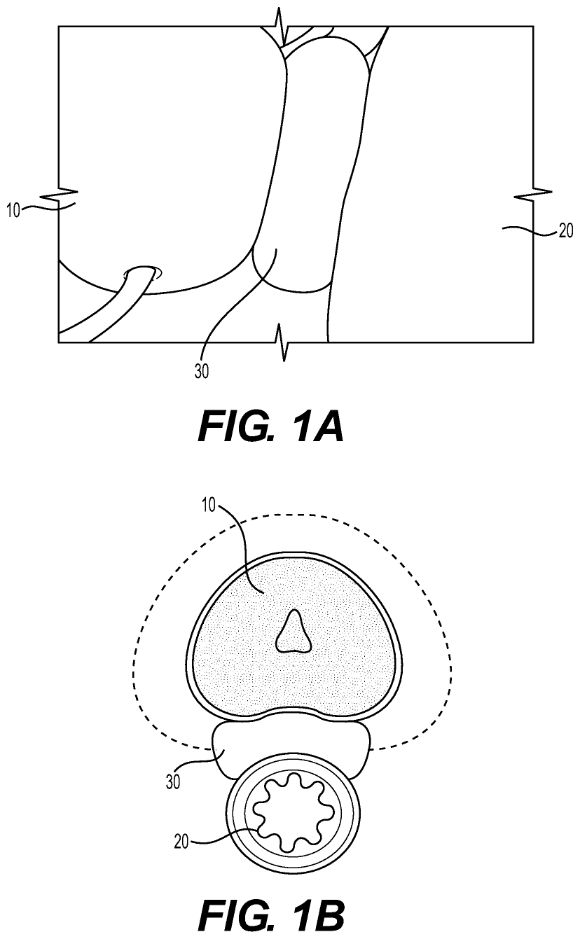

Turning to the drawings, FIG. 1A is a perspective view and FIG. 1B is a partial cross-section view illustrating example filler 30, in the form of a gel composition having been delivered by the mixing system of this disclosure between rectum 20 and prostate 10 of a patient in Denonvilliers' space.

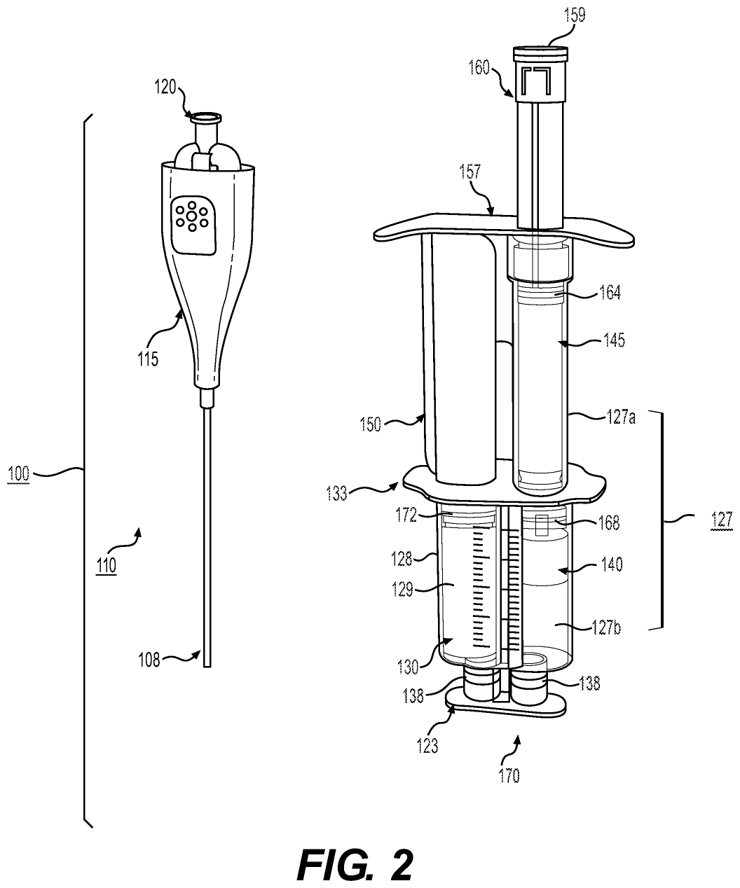

FIG. 2 shows an exploded view of an exemplary mixing system 100 in accordance with certain aspects of the present disclosure for mixing a gel composition. The system 100 can include a needle assembly 110 attachable to the main assembly 170 of system 100. Needle assembly 110 can include needle 108, which can be any needle of this disclosure suitable for hydrodissection as well as delivering filler 30 (e.g., the gel composition) to the treatment site. A proximal end of needle 108 can be connected to a distal end of a connector 115.

Main assembly 170 of system 100 can include a multi-lumen chamber formed by a first lumen 127 inside a first barrel and a second lumen 129 inside a second barrel. Each lumen 127, 129 can be oriented parallel with the other, running side-by-side. Lumen 127 can be divided into a proximal portion 127a and a distal portion 127b. A first plunger stopper 164 can be located at a distal end of a first plunger rod 160. Rod 160 can be advanceable within lumen 127 and include the first plunger stopper 164 at a distal end of rod 160. Rod 160 can be advanced by button 159 positioned on a proximal end of rod 160. A second plunger stopper 168 can be positioned within lumen and separate portions 127a, 127b. Portions 127a and 127b can include one or more constituents (e.g., a fluid, liquid or otherwise). Distally moving rod 160 can cause stopper 164 to advance constituent(s) of portion 127a so as to open a barrier associated with stopper 168 thereby allowing constituent(s) of each portion 127a, 127b to intermix and form precursor. As used herein, the term “fluid” is defined broadly and can include liquids, gels and particulate matter such as granules, pellets, or powders, or any combination of liquids, gels, oils, and/or particulate matter (e.g., granules, pellets, or powders). In some examples, constituent(s) of portion 127a can be a constituent 145 (e.g., a fluid such as diluent) and, in some examples, at least one of the constituents of portion 127b can include constituent 140 (e.g., an activating agent, such as PEG or any other agent mixable with constituent 145 to intermix and form precursor 145′). The diluent can be a branched polymer having a plurality of succinimidyl termini dissolved in a low pH (4.0) containing a low molecular weight precursor comprising nucleophiles, though other diluent fluid solutions are contemplated within the scope of this disclosure. Once mixed together, precursor 145′ can be formed in portion 127b.

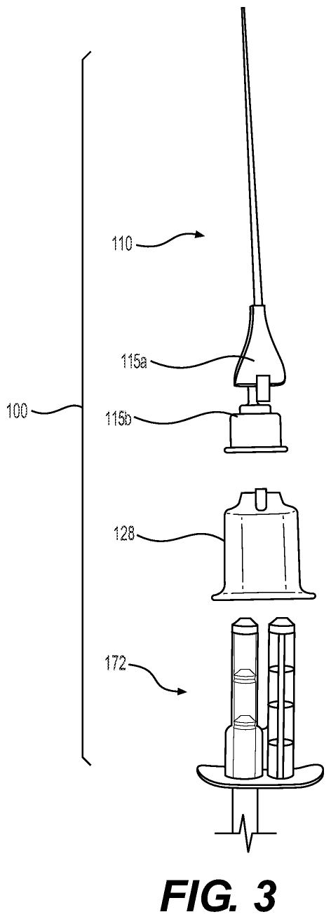

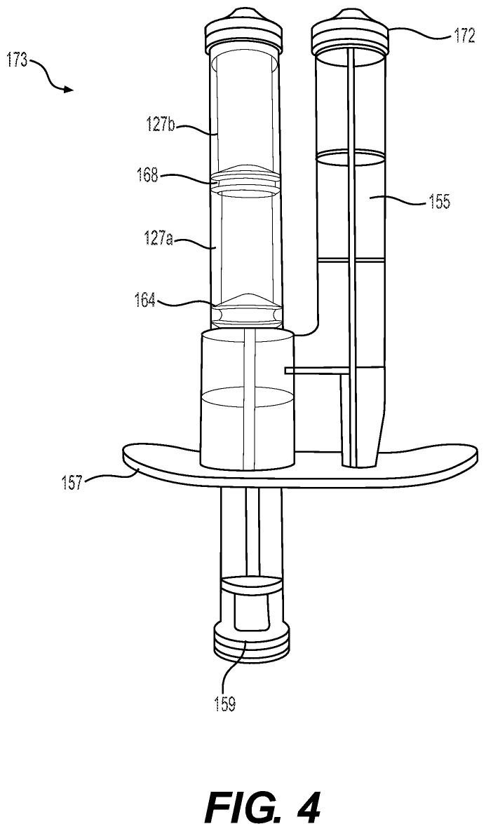

Lumen 129 can similarly include a plunger rod 155 slidable therein. A distal end of rod 155 can include a stopper 172. A proximal end of rod 155 can include an actuating flange 157 configured so that a user can a press thereon to drive rod 155 proximally or distally. As seen clearly in FIG. 4, plunger rod 155, flange 157, rod 160, and portions 127a, 127b can be partially or entirely integrally formed together to form plunger assembly 173. Turning back to FIGS. 2 and 3, assembly 170 can be assembled to form lumen 129 by positioning distal ends of rods 155, 160 proximate distal ends of receiver 128. Receiver 128 can include an open proximal end with a flange 133 while the distal ends of receiver 128 can include a plurality of smaller openings configured to receive ports 138 of assembly 170. This is shown more clearly in FIG. 3, which illustrates an exploded view of system 100, including plunger assembly 173 proximal and just prior to being assembled through the open upper end of receiver 128.

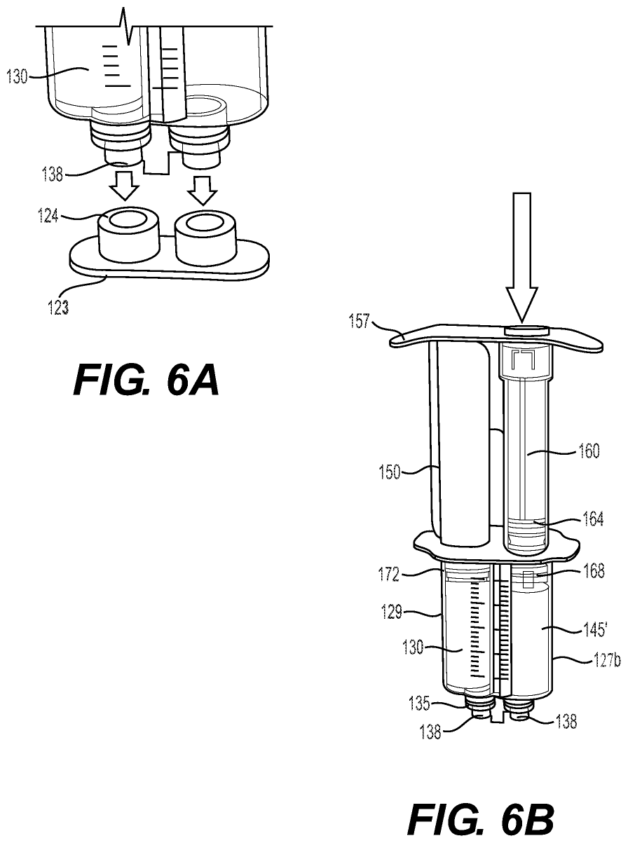

Each of ports 138 are configured to permit egress of fluids from respective lumens 127, 129. Optionally, as shown in FIG. 2, system 100 can include a detachable cap 123 so as to seal ports 138 between uses or during transit when stored in separate packaging or a kit. Also optionally, in a first state before mixing, the system 100 can include a retainer 150 removably positioned between flanges 133 and 157 so as to prevent unwanted movement of rod 155. In some aspects, once assembly 173 is nested within receiver 128, lumen 129 is formed between an outer surface of portion 127b and an inner surface of receiver 128. Constituent 130 (e.g., accelerant fluid) can be positioned therein, as shown clearly in FIGS. 2 and FIGS. 6A-6B, and stopper 172 of rod 155 can advance constituent 130 to mix with precursor 145′ once distal of ports 138. In some aspects, while flange 157 is permanently or temporarily attached to button 159 of rod 160, distally advancing flange 157 can distally advance both stopper 172 as well as rod 160, stopper 164, and/or stopper 168 so that the precursor 145′ and constituent 130 are capable of egressing through respective ports 138 and mixing together distal thereof (e.g., in connector 115).

In some aspects, flange 157 can include an opening sized to permit rod 160 to slide therethrough. However, button 159 can be larger than the opening so as to prevent button 159 from sliding distal of flange 157 and ensure that once button 159 and flange 157 are aligned or otherwise attached, flange 157 being distally advanced can drive both rod 160 and rod 155 simultaneously.

Turning back to needle assembly 110, in FIGS. 2-3 and 5, it can be seen that connector 115 includes a distal portion 115a and a proximal portion 115b. Portion 115b can be insertable into an open proximal end of portion 115a to nest therewith and form connector 115. Portion 115a can be substantially hollow with a tapered or Y-shape profile for its outer surface. Portion 115a can terminate in a distal end with a mixing lumen 117 running therethrough.

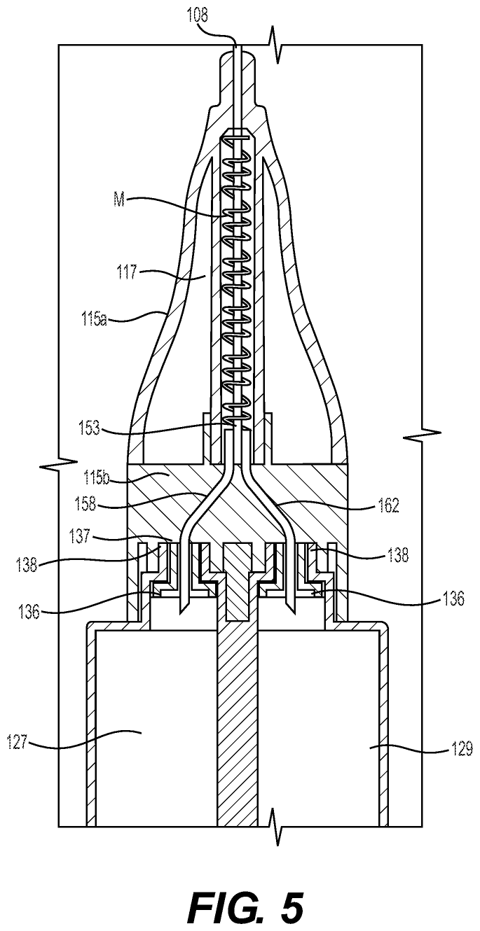

As seen more clearly in FIG. 5, which is a partial cross-section view of connector 115 assembled with a distal end of assembly 170, each of lumens 127, 129 can be in fluid communication with a proximal end of lumen 117 of connector 115. Portion 115b can be substantially solid with a fluid path 137 running from port 138 of each lumen 127, 129 to a proximal end of lumen 117. Lumen 117 can include a static mixer 153 so that fluid from respective lumens 127, 129 can mix together and form the gel composition to be delivered through needle 108.

In some aspects, portion 115b can include a tube 158 (e.g., a hypotube) with a proximal end configured in fluid communication with lumen 127 and pierce a corresponding membrane or seal 136 of port 138. Portion 115b can also include tube 162 (e.g., a hypotube) with a proximal end configured in fluid communication with lumen 129 and pierce a corresponding membrane or seal 136 of port 138. In this respect, once precursor 145′ is in position in lumen 127 and constituent 130 is positioned in lumen 129 with air purged from each and connector 115 assembled thereto, distally moving rod 155 can cause precursor 145′ and constituent 130 to egress through respective ports 138 and respective tubes 158, 162 to mix with each other in lumen 117. Tubes 158, 162 can form a Y-shape, as in FIG. 5, though any other shape can be used as needed or required.

Now, turning to FIGS. 6A-9B are example steps of a process of using system 100 according to certain aspects of this disclosure. While certain steps are shown as a sequence between each figure, in other embodiments, fewer steps are contemplated and the order by which steps are performed can be different than what is illustrated. In FIG. 6A, system 100 is introduced and cap 123, including each of its receivers 128, can be detached from ports 138.

With cap 123 removed, in FIG. 6B system 100 is illustrated in a first state with rod 155 fully retracted and retainer 150 positioned between flanges 133, 157. In the depicted configuration, rod 155 is incapable of distally moving as a result of retainer 150 being wedged between flanges 133, 157. In contrast, rod 160 has been advanced distally as denoted by the downward arrow so that constituent 145 has been moved by stopper 164 and barrier of stopper 168 moved causing constituent 145 to mix with constituent 140.

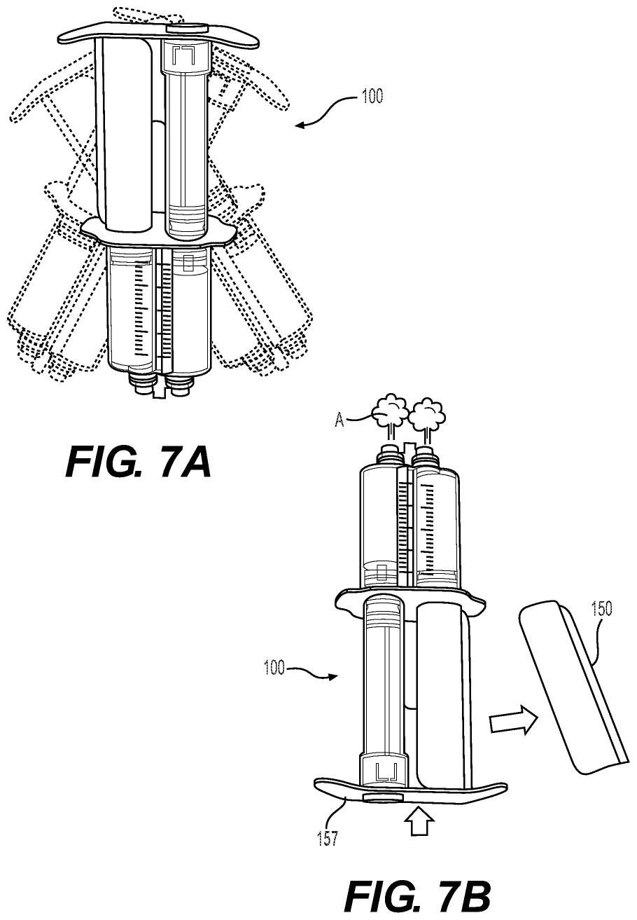

In FIG. 7A, system 100 can be shaken back and forth to ensure precursor forms as a result of mixing between constituent 145 and constituent 140, while constituent 130 remains in lumen 129. Preferably, the shaking action of FIG. 7A is done while the ports 138 are oriented generally upward. However, the shaking to effect proper mixing of precursor 145′ can be performed in other orientations (e.g., generally downward, etc.), as needed or required.

In FIG. 7B, with precursor 145′ formed and constituent 130 in lumen 129, retainer 150 can be removed and flange 157 can be distally advanced slightly to purge any air A from system 100 out through respective ports 138. Preferably, air A is purged as shown while system 100 is oriented generally upward.

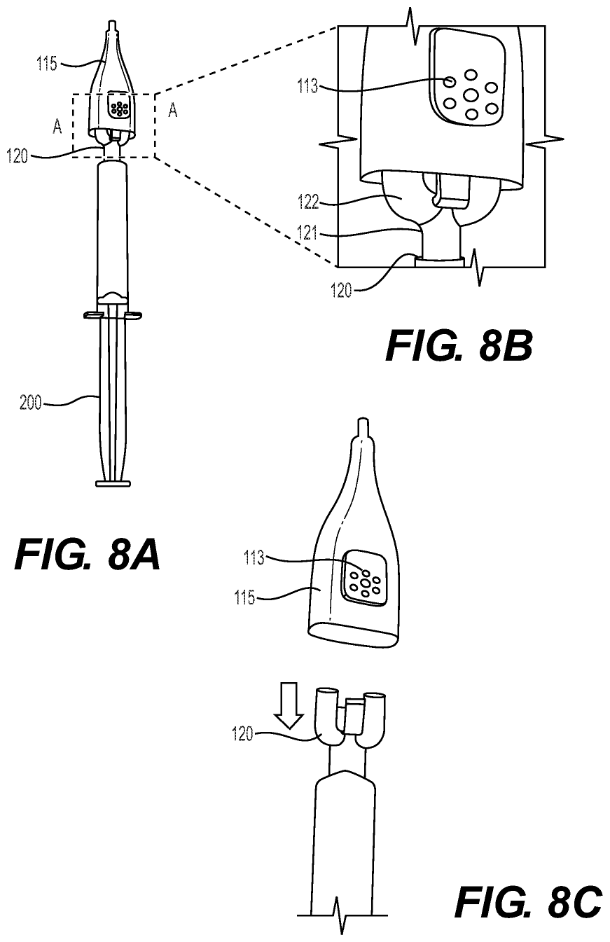

Separately, in FIG. 8A, connector 115 is shown connected to syringe 200 via adaptor 120, as illustrated more clearly in FIG. 8B, which is a close-up of section A-A. Adaptor 120 can provide a fluid bridge 122, 121 from each of tubes 158, 162 of connector 115 to syringe 200. Adaptor 120 can couple to syringe 200 with a luer fitting or any other connector operable to connect with a distal end of syringe 200. While not shown, during use connector 115 is contemplated to be connected to needle 108 for hydrodissection at the treatment site with saline from the syringe 200.

After hydrodissection with syringe 200, needle 108 and adaptor 120, and syringe 200 can be released from connector 115, as shown in FIG. 8C. Connector 115 can include an externally positioned button 113 to open and close corresponding connecting latches of connector 115. However, other coupling approaches between connector 115, adaptor 120, and syringe 200 are contemplated as needed or required. For example and without limitation, snap fit connectors, magnetic connectors, female—male connectors, hook and loop fasteners and the like are contemplated.

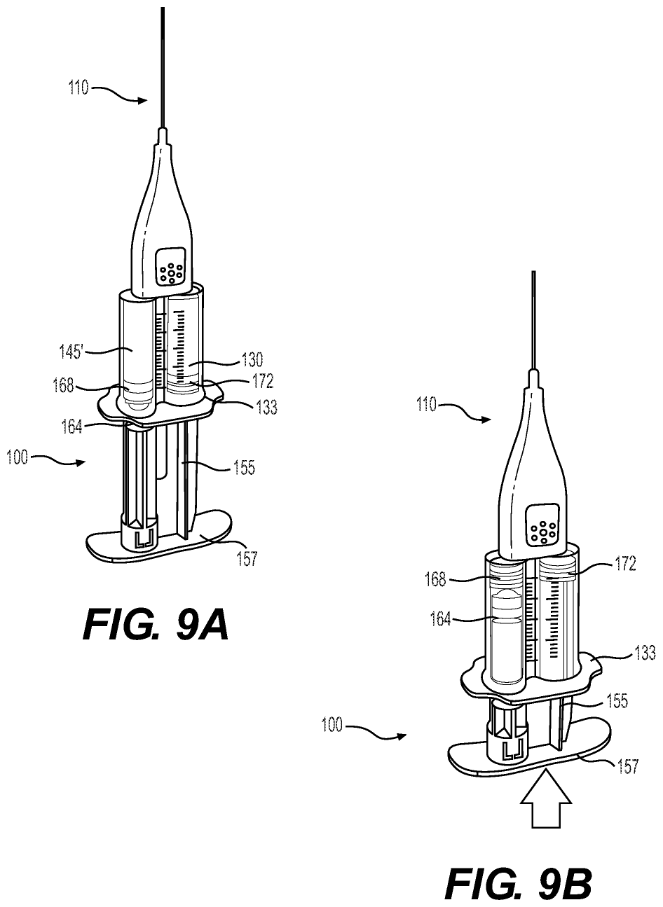

Turning to FIG. 9A, system 100 is now connected to connector 115, which remains primed and connected to needle 108 in position at the treatment site. With system 100 ready and connector 115 primed, in FIG. 9B a user can advance flange 157 distally so that corresponding rods 155, 160 distally drive respective stoppers 164, 172 and advance precursor 145′ from lumen 127 and constituent 130 from lumen 129, through ports 138, and into the mixing lumen 117 of connector 115. As long as flange 157 continues advancing, precursor 145′ and constituent 130 can mix within mixing lumen 117 and continue egressing through needle 108 and ultimately to the treatment site. Optionally, lumen 117 can include a static mixer M configured to thoroughly mix the fluids together to form the gel composition to be delivered to the treatment site. System 100 as shown is relatively easy to assemble and minimizes potential unintentional gel mixing errors prior to delivery.

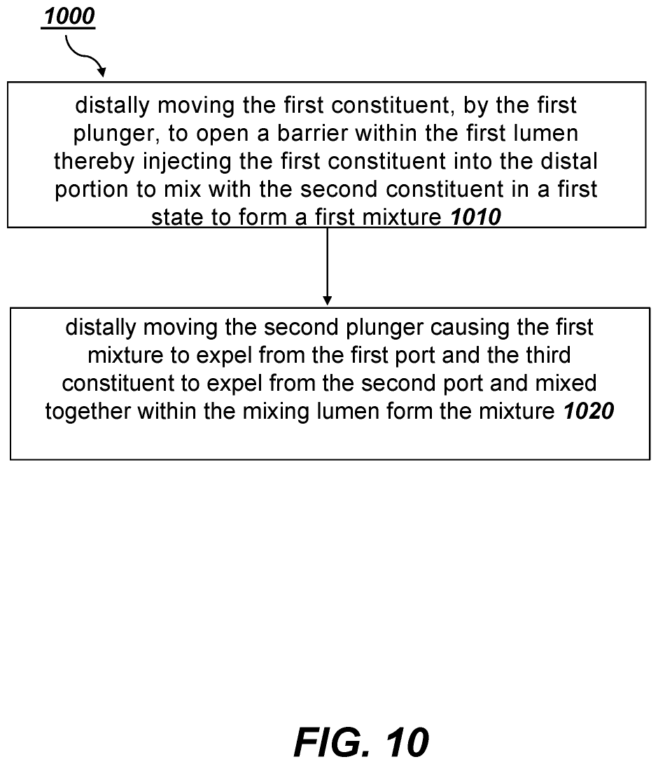

FIG. 10 depicts a method 1000 of any of the herein disclosed mixing systems. Step 1010 of method 1000 can include distally moving the first constituent, by the first plunger, to open a barrier within the first lumen thereby injecting the first constituent into the distal portion to mix with the second constituent in a first state to form a first mixture. Step 1020 of method 1000 can include distally moving the second plunger causing the first mixture to expel from the first port and the third constituent to expel from the second port and mixed together within the mixing lumen to form the mixture. Method 1000 can end after step 1020. In other embodiments, additional steps according to the examples described above can be performed.

Other aspects and embodiments of the present disclosure will be apparent to those skilled in the art from consideration of the specification and practice of the embodiments disclosed herein. While certain features of the present disclosure are discussed within the context of exemplary procedures, the compositions, systems, and methods may be used for other medical procedures according to the general principles disclosed. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein. It is intended that the specification and examples be considered as exemplary only, with a true scope and spirit of the present disclosure being indicated by the following claims.

What is claimed is:

1. A system for producing a mixture to deliver to a treatment site, comprising:
a mixing lumen;
a multi-lumen chamber connected to a proximal end of the mixing lumen comprising a first lumen aligned and adjacent a second lumen;
the first lumen configured to comprise a first constituent in a proximal portion of the first lumen and a second constituent in a distal portion of the first lumen, a first plunger internally positioned within the first lumen to distally move the first constituent into the distal portion to mix with the second constituent in a first state to form a first mixture, the first lumen terminating in a first port; and
the second lumen configured to comprise a third constituent, a second plunger internally positioned within the second lumen to distally move the third constituent and the first mixture, and the second lumen terminating in a second port;
wherein distally moving the second plunger causes the first mixture and the third constituent to be delivered through the first and second ports and mixed together within the mixing lumen to form the mixture.

2. The system of claim 1, further comprising:
a connector comprising the mixing lumen and being attachable to a distal end of the multi-lumen chamber and a proximal end of a needle;
wherein the system is configured to deliver the mixture through the mixing lumen and the needle.

3. The system of claim 2, wherein the connector comprises a greatest width adjacent a distal end of the multi-lumen chamber, and wherein at least one of the first port and the second port comprises an air-permeable fluid-impermeable membrane.

4. The system of claim 2, the connector further comprising:
a first tube configured to be in fluid communication with the first port and the mixing lumen of the connector; and
a second tube configured to be in fluid communication with the second port and the mixing lumen of the connector.

5. The system of claim 1, wherein proximal and distal ends of the first and second lumens are aligned with each other in a second state.

6. The system of claim 1, wherein the first constituent is a diluent and the third constituent is an accelerator.

7. The system of claim 1, wherein the first mixture is a precursor fluid solution.

8. The system of claim 1, wherein the proximal and distal portions of the first lumen are separated by a barrier; and
wherein distally moving the first plunger causes the barrier to open so the first constituent mixes with the second constituent in the first state to form the first mixture.

9. The system of claim 1, wherein the proximal portion of the first lumen, the first plunger and the second plunger are integrally formed in a plunger assembly.

10. The system of claim 9, wherein the first plunger is configured to advance within the first lumen independent of the second plunger.

11. The system of claim 1, wherein in a first state, the system further comprises a retainer removably positioned between a flange of the second plunger and the multi-lumen chamber so as to prevent movement of the second plunger; and
wherein, in a second state, the retainer is removed so that the second plunger is capable of distally moving the second plunger to cause at least one of the first mixture and the third constituent to egress through a respective lumen and mix together in the mixing lumen to form the mixture.

12. A connector for mixing and delivering a mixture to a treatment site, comprising:
a distal end configured to be attached to a needle;
a proximal end being wider than the distal end;
a mixing lumen between the proximal and distal ends and extended proximally from the distal end and configured to be in fluid communication with a lumen of a needle;
a first tube proximally extended from a proximal end of the mixing lumen to a first lumen receiver; and
a second tube proximally extended from the proximal end of the mixing lumen and opposite the first tube to a second lumen receiver.

13. The connector of claim 12, wherein the first and second tubes form a Y-shape.

14. The connector of claim 12, wherein the mixing lumen comprises a static mixer.

15. The connector of claim 12, wherein an outer surface of the connector is tapered between proximal and distal ends.

16. The connector of claim 12, wherein a proximal end of the mixing lumen is positioned centrally within an inner diameter of the connector between the proximal end distal ends of the connector.

17. A method for producing a mixture with a mixing system to deliver to a treatment site, the mixing system comprising a mixing lumen and a multi-lumen chamber connected to a proximal end of the mixing lumen comprising a first lumen aligned and adjacent a second lumen;
the first lumen configured to comprise a first constituent in a proximal portion of the first lumen and a second constituent in a distal portion of the first lumen, a first plunger internally positioned within the first lumen and terminating in a first port; and
the second lumen configured to comprise a third constituent, a second plunger internally positioned within the second lumen and terminating in a second port, the method comprising:
distally moving the first constituent, by the first plunger, to open a barrier within the first lumen thereby injecting the first constituent into the distal portion to mix with the second constituent in a first state to form a first mixture; and
distally moving the second plunger causing the first mixture to expel from the first port and the third constituent to expel from the second port and mixed together within the mixing lumen to form the mixture.

18. The method of claim 17, further comprising:
connecting a primed connector and a needle to a distal end of the multi-lumen chamber, the primed connector comprising the mixing lumen and being a Y-shaped connector, the method further comprising:
positioning a first tube in fluid communication with the first port and the mixing lumen of the primed connector; and
positioning a second tube in fluid communication with the second port and the mixing lumen of the primed connector.

19. The method of claim 17, wherein the proximal portion of the first lumen, the first plunger and the second plunger are integrally formed in a plunger assembly, the method further comprising:
positioning the second lumen and the distal portion of the first lumen within a lumen receiver; and
at least partially positioning the first and second plungers within the lumen receiver in a second state.

20. The method of claim 17, wherein the first constituent is a diluent and the third constituent is an accelerator, and wherein the first mixture is a precursor fluid solution.
